(19) United States
(12) Patent Application Publication
(10) Pub. No.: US 2002/0051116 A1
(43) Pub. Date: May 2, 2002

(54) Eye tracker for refractive surgery

(76) Inventors: Paul Phillip Van Saarloos, Karrinyup (AU); Natalie Marguerita Taylor, Berlin (DE)

Correspondence Address: BAKER BOTTS LLP, 44TH FLOOR, 30 ROCKEFELLER PLAZA, NEW YORK, NY, 10112-0228, US

(21) Appl. No.: 09/849,015

(22) Filed: May 4, 2001

Related U.S. Application Data
(63) Continuation of application No. PCT/AU99/00978, filed on Nov. 8, 1999.

(30) Foreign Application Priority Data
Nov. 6, 1998 (AU) PP6973

(51) Int. Cl. A61B 3/10

(52) U.S. Cl. 351/204

(57) ABSTRACT

A method of determining the position or attitude of an eye, including the steps of directing a plurality of eye-safe light beam components onto the eye, said components defining an area of incidence on the eye substantially larger than the pupil; receiving an image of light thereafter reflected by the eye; analyzing the image by identifying which of the light beam components produces a bright eye reflection in said image; and determining the position or attitude of the eye by further analysing the image on the basis of said identification.

### FIELD OF THE INVENTION

The present invention relates generally to determining the position or attitude of an eye, and in a particular though not exclusive application is concerned with an optical aiming system for tracking movements of an eye, particularly during ophthalmic laser surgery. This invention has an application for use in operations for the refractive correction of the eye, such as Photorefractive Keratectomy (PRK) and Laser-in-situ Keratomileusis (LASIK). The present invention will be described in terms of this application, though it may also be applied to other eye surgery, eye tracking or gaze analysis applications.

### BACKGROUND ART

It is known in the art that removing tissue from specific areas of the cornea can correct common visual defects such as myopia, hyperopia and astigmatism. Reshaping the curvature of the cornea and altering the power of the eye enables the eyeball to better bring light rays into focus. Operations such as Photorefractive Keratectomy (PRK), Laser-in-situ Keratomileusis (LASIK) and intrastromal ablation, utilise laser energy to ablate minute portions of corneal tissue. Currently, refractive surgery is performed by the excimer laser, operating at a wavelength of 193 nanometers. Solid state frequency converted Neodymium and Erbium doped lasers also have potential for use in this area.

For operations such as PRK to be successful, laser exposure must be confined to a target region on the cornea. No significant quantity of laser energy should penetrate below a certain depth or deviate outside the treated area. Gross movements of the eye, head or the laser equipment, or involuntary saccadic eye movements, may cause decentration of the surgical target region. Such a momentary lapse might result in the laser beam straying outside the treatment zone. Under- or over-correction, or uneven ablation patterns, with resultant post-operative astigmatism, glare or halos at night, may be the consequences of such a loss of fixation.

Thus, care must be taken to prevent radiation from deviating outside the target region on the cornea during refractive surgery. A patient's eye movements should therefore be adequately constrained. A common method of maintaining a stable eye position is to urge the patient to fixate their gaze on a flashing light spot generated by a light emitting diode (LED) located under the surgical microscope of the laser system. This method usually reduces gross eye movements, but involuntary saccadic and other movements may still occur. It also relies on the reaction time of the operating surgeon to judge and respond before a stray shot is fired.

Physical grasping devices and suction rings have been used in another approach to maintain a fixed eye position. U.S. Pat. No. 4,665,913 to L'Esperance first described a scanning laser system that utilises a head and eye restraining clamp to prevent movement during surgery. U.S. Pat. No. 5,360,424 describes an alternative system for precisely aligning a laser beam to a target region. This patent teaches the use of an eye restraining device, such as an eye cup secured by suction. A floating optic is coupled to the securing element as part of the imaging system of the laser. This optic directs the laser to follow any minor movements of the target tissue. Nevertheless, the pressure exerted by such restraining devices has the ability to cause discomfort to the patient, and to distort the shape of the eyeball, resulting in an unpredictable surgical outcome.

Non-contact devices which are able to automatically detect the position of the eye relative to the surgical laser beam are a preferred option for tracking eye movements in ophthalmic laser surgery. Reflections from the cornea and the pupil are known to move in proportion with eye movements, making them good candidates for eye tracking. Tracking devices which use reflections from the surfaces of the eye have been developed to this end.

One existing method involves following the position of the first and fourth Purkinje reflections (See U.S. Pat. Nos. 3,712,716, 3,724,932 and 4,287,410). Under infra-red illumination, undetectable by the human eye, images of the Purkinje reflections can be observed, the first of which is the reflection from the cornea, the corneal glint, and the fourth, the reflection from the back of the lens. These reflections are useful for tracking rotational movements of the eye. The eye tracker described in U.S. Pat. No. 3,724,932 measures the spatial separation of the first and fourth reflections, using an illuminated, rotating, scanning disc with orthogonal slits. The Purkinje reflections are recorded by a photomultiplier tube. Monitoring the position of the images in relation to the photomultiplier can give an indication of the spatial separation of the Purkinje reflections, and therefore, the position of the optical axis of the eye can be infra-red.

U.S. Pat. No. 4,848,340 describes an alternative eye tracking device that utilises reflections to monitor reference marks scribed onto the surface of the eye. The patient is required to fixate on a visual reference which has a known relationship with the axis of the surgical laser beam. In this way, the eye's optical axis is co-axially aligned with the laser beam's axis. The surgical laser is then used to mark a grid pattern onto the cornea. Infra-red light is used to illuminate the grid, and its reflections are recorded by a sensor, which detects any movement of the grid from its original alignment. A variation in the intensity of light received from points on the grid indicate that eye movement has occurred. An error signal is then generated and transmitted to a guidance system, which in turn steers the laser beam to compensate and realign the optical axis.

Autonomous Technologies have also developed an eye tracker, described in U.S. Pat. Nos. 5,632,742 and 5,442,412, specifically for use with a scanning excimer laser during refractive surgery. This device utilises a polarised, near infra-red light source of approximately 900 nm, delivered to the eye-to-be-treated as a plurality of light spots. In order to centre the tracking device a mark is initially made in the centre of the pupil by a blunt needle. The light spots are then aimed at either a natural or man made boundary on the surface of the eye, this boundary being incident with eye movement. Such a boundary may include the pupil/iris border or the iris/sclera boundary. Alternatively an ink ring or a tack may be used. The energy reflected from the light spots hitting the cornea is detected through an infra-red detector. Any change in the reflected energy at one or more of the light spot positions indicates that movement of the eye has occurred. This feedback can be employed to control the drivers used to position the laser, or to trigger an alarm, in cases where an excessively large movement has been detected.

The methods described above may require otherwise unnecessary, invasive procedures to the cornea. Eye movement detection has also been an important field of research in areas such as fitness testing, photography, infant research, communication and disability support. Eye trackers applied to these other fields of study have provided a non-invasive means to determine the direction of eye gaze.

Eye gaze provides a stable means of communication and may be useful in a computer interface utilising eye movements instead of keystrokes. A method of feature extraction has been developed to track eye gaze in potential computerised disability support systems. Ebosawa and Amano, SICE '94:985-990 (1994) and Tomono, lida and Kobayashi, The Proceedings of SPIE: Optics, Illumination and Image Sensing for Machine Vision IV, 1194, 2-12 (1989) (see also U.S. Pat. No. 5,016,282) present similar methods of pupil detection. Eye position is determined by the relative positions of the pupil centre and the first Purkinje reflection, the corneal glint.

In the method described in U.S. Pat. No. 5,016,282, infra-red light sources and a video camera are utilised to extract features necessary to determine if eye movement is occurring. Two near infra-red light sources (such as LEDs operated at 850 nm and 900 nm) and an infra-red sensitive video camera, are used to obtain images with different brightness levels. The LEDs may be driven with an electronic shutter to reduce the amount of light exposure to the eye. Feature points with different brightness levels are extracted in separate images. The feature points are emphasised against the background noise by subtracting consecutive images from one another.

Using the above method, one illumination source is positioned coaxial to the camera, the other is slightly off axis. The light from the coaxial source enters the pupil and is reflected from the retina, producing a bright disc of illumination at the pupil. The rays from the source that is off axis are not reflected through the pupil, resulting in a dark pupil emphasised against the lighter background. Under both “bright eye” and “dark eye” conditions, some of the light is also reflected off the cornea, resulting in an image of the corneal glint. Owing to the optical properties of the cornea, the light from the corneal glint is polarised, while the reflections from the pupil and other surfaces are unpolarised. Tomono et al. (1989) suggest that perpendicularly polarised infra-red rays and a polarising filter in front of the detecting device will aid in corneal glint detection. The filter then blocks one of the polarised reflections, to produce images with or without a corneal reflection.

The relative positions of the pupil and the corneal reflection can then be used to determine the direction of eye gaze. Image processing apparatus in a personal computer processes the images. The “bright eye”, “dark eye” and the “glint” and “no glint” images can be subtracted to obtain a difference image that accentuates the positions of the pupil and the glint against the background. Tomono et al (1989) calculated the centre of gravity of the pupil, or the pupil centre, and used it, in combination with the position of the cornea glint, to indicate the direction of gaze.

However, owing to an incompatible central IR source alignment approach, and an inadequate range of movement of the eye at the required working distance over which the source illuminates the pupil, the technique of Tomono et al is unsuitable for ophthalmic surgery applications.

It is therefore an object of the present invention to provide a non-invasive method and apparatus for determining eye position which facilitates tracking of eye movement in real time, and which is thereby adaptable to ophthalmic laser surgery for the correction of refractive errors of the eye, for controlling the laser source to compensate for eye movement.

### SUMMARY OF THE INVENTION

Thus, according to a first aspect of the present invention there is provided a method of determining the position of the pupil of an eye, including the steps of:

directing an eye-safe light beam having a plurality of components onto the eye, said components defining an area of incidence for the beam on the eye substantially larger than the pupil of the eye;

receiving an image of light thereafter reflected by the eye;

analysing the image by identifying which of the light beam components produces a bright eye reflection in said image;

determining the position of the pupil by further analysing images on the basis of said identification.

The plurality of light beam components may be a device comprising a pair of crossed lines, or a grid of intersecting lines, or an array of light spots.

Preferably, the method further includes generating the eye-safe light beam having a plurality of light beam components by splitting an initial light beam into the components. Preferably, the initial light beam and light beam components are collimated.

The method may further include directing at least one further eye-safe light beam onto the eye from a lateral direction, and receiving a further dark-eye image of light thereafter formed by reflection of said further light beam(s) by the eye, wherein said further analysis includes subtracting or otherwise comparing said images.

Preferably, the method further includes recording the image(s).

Preferably, the method includes repeating the aforesaid steps to monitor changes in the position of the pupil of the eye.

In its first aspect, the invention extends to a method of controlling the aim of a surgical laser including determining the position of the pupil of an eye according to the aforedescribed method, and further including controlling the aim of the laser in response to the determined position.

The eye-safe beam is preferably a beam of infra-red light.

The invention further provides, in its first aspect, apparatus for determining the position of the pupil of an eye, including means for directing an eye-safe light beam having a plurality of components onto an eye, the components defining an area of incidence on the eye substantially larger than the pupil of the eye. Further included are means for receiving an image of light thereafter reflected by the eye, and means for analysing the image by identifying which of the light beam components produces a bright eye reflection in the image, and for further analysing the image on the basis of such identification, whereby to determine the position of the pupil.

The invention further provides surgical laser apparatus including laser means for producing a beam of ablative radiation and aiming the beam at an eye, and apparatus as aforedescribed for determining the position of the pupil of the eye and for controlling the aim of the beam of ablative radiation in response to the determined position.

In a second aspect, the invention provides apparatus for facilitating determination of the position of the pupil of an eye, including:

means for generating an eye-safe light beam;

optical means disposed to receive said light beam for splitting it into a plurality of light beam components to be directed onto an eye, said components defining an area of incidence for the beam on the eye substantially larger than the pupil of the eye; and

means for receiving an image of light thereafter reflected by the eye;

wherein said light beam defines a device comprising one or more devices selected from a pair of crossed lines, a grid of intersecting lines, and an array of light spots that respectively form said plurality of components.

The analysis method may include transforming a difference image obtained from first and second images from greyscale into black and white, and/or calculating a centre of mass of the remaining blob to calculate x and y co-ordinates and find the centre of the pupil of the eye.

The method may include performing edge detection and/or fitting a circle or ellipse to find the centre of gravity of an extracted feature.

The present invention still further provides an apparatus for determining the position of the pupil of an eye during refractive surgery so that the aim of an ablative laser can be adjusted accordingly, including:

laser means for producing a beam of ablative radiation;

a first infra-red light source for illuminating the pupil of said eye;

an optical system for directing said first infra-red source;

a second infra-red light source for illuminating the iris of said eye;

focussing means for focussing light reflected from surfaces of the eye;

recording means for recording images of the light reflected from said surfaces;

image processing means for determining the position of the pupil of the eye; and

controller means for interpreting said images and directing said laser to the appropriate position on the cornea in accordance with the determined position of the pupil.

Preferably there may be digitising means for converting recorded video images to digital images, which digitising means may include frame grabbing means, eg a frame grabber card.

The laser means may be eg a 193 nm excimer laser, a 213 nm or 3 micron solid state laser or any other suitable ablative laser for reshaping the surface of the cornea, or a short pulsed near infra-red or visible laser suitable for intrastromal ablation.

The surgery to which the operation and method may be usefully applied includes surgery for the correction of refractive errors of the eye, such as in Photorefractive Keratectomy (PRK), Laser-in-situ Keratomileusis (LASIK) or intrastromal ablation.

Preferably, the main source of eye-safe light is an infra-red light source, eg an infra-red light, an infra-red LED, a low power laser diode or a light with an infra-red filter, directed from a direction approximately co-linear with the surgical laser beam.

Preferably the further eye-safe light source is a broad illumination infra-red light, such as an infra-red LED or bulb with IR filter.

Preferably the further eye-safe light source is a ring of LEDs.

Preferably all light sources provide near infra-red illumination in the range 780 nm to 1000 nm.

Preferably the main and further infra-red light sources use different infra-red illumination wavelengths, which can preferably be separated with band pass filters.

Preferably the image receiving means is a charge-coupled device camera or an infra-red camera.

Preferably the analysing means and controller comprise one or more personal computers or a microprocessors.

Preferably said analysing means includes image processing means which advantageously includes a machine coded or software coded algorithm that detects the pupil from the bright eye effect of light entering the pupil.

The invention also provides method for determining the position of the pupil of an eye during ablative laser surgery so that the aim of the ablative laser can be adjusted accordingly, including:

directing a first beam of infra-red light from a first position onto said eye;

directing at least one second beam of infra-red light from a second position onto said eye through an optical system;

imaging light from said first and second beams reflected from said eye to form first and second images respectively; and

comparing said images to determine said position of the pupil of said eye.

In order that the present invention may be more fully ascertained, preferred embodiments will now be described, by way of example, by reference to the accompanying drawings, in which:

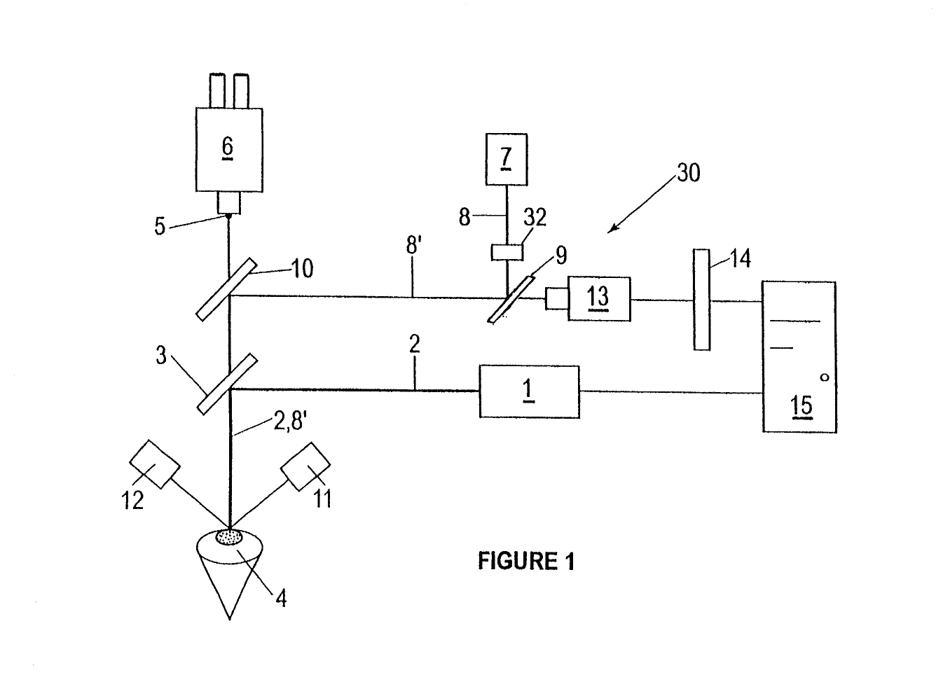

FIG. 1 is a schematic view of the principal relevant components of ophthalmic surgery apparatus incorporating an eye-tracking device according to a first preferred embodiment of the present invention;

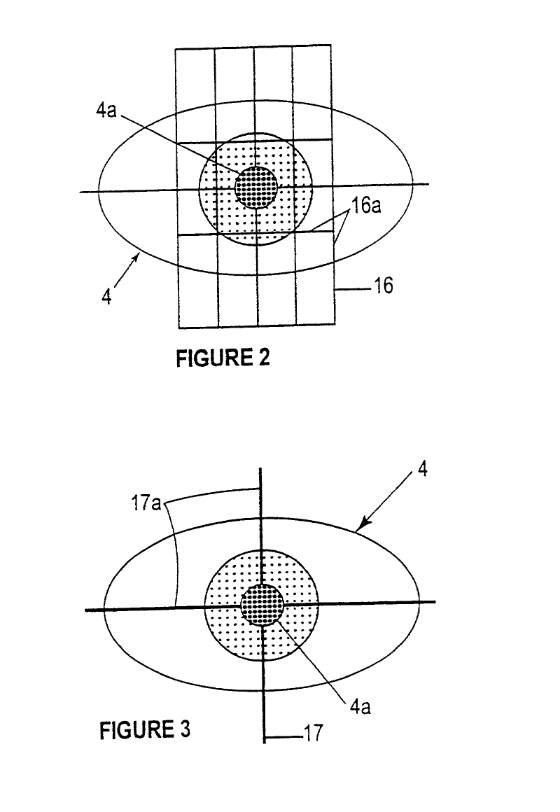

FIGS. 2, 3 and 4 depict alternative structured light beams having plural components, shown projected onto an eye;

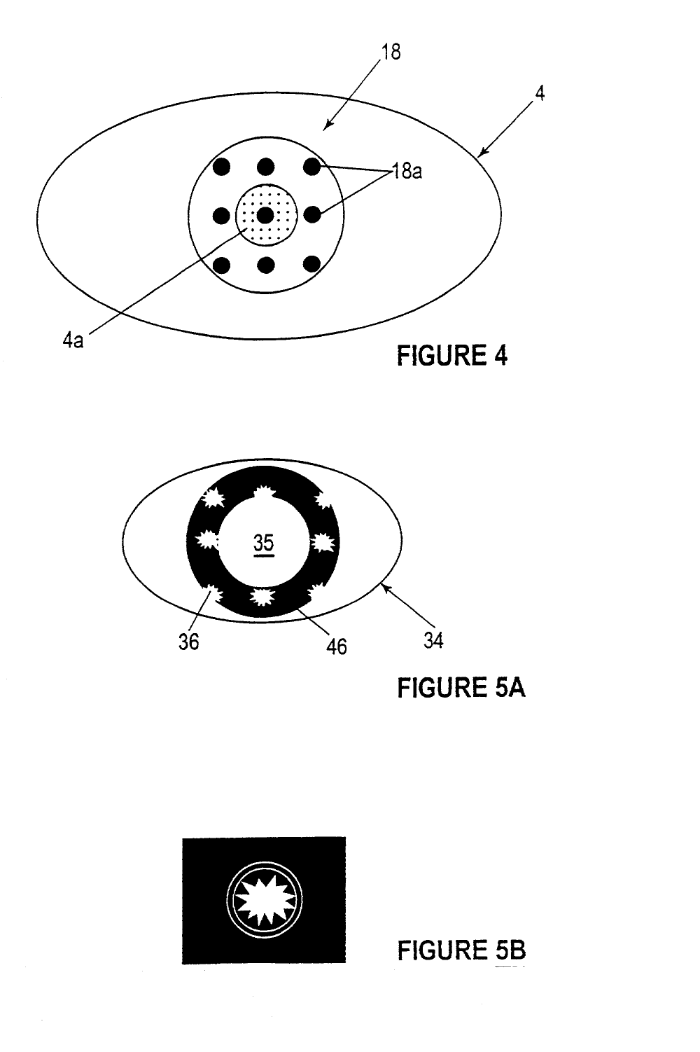

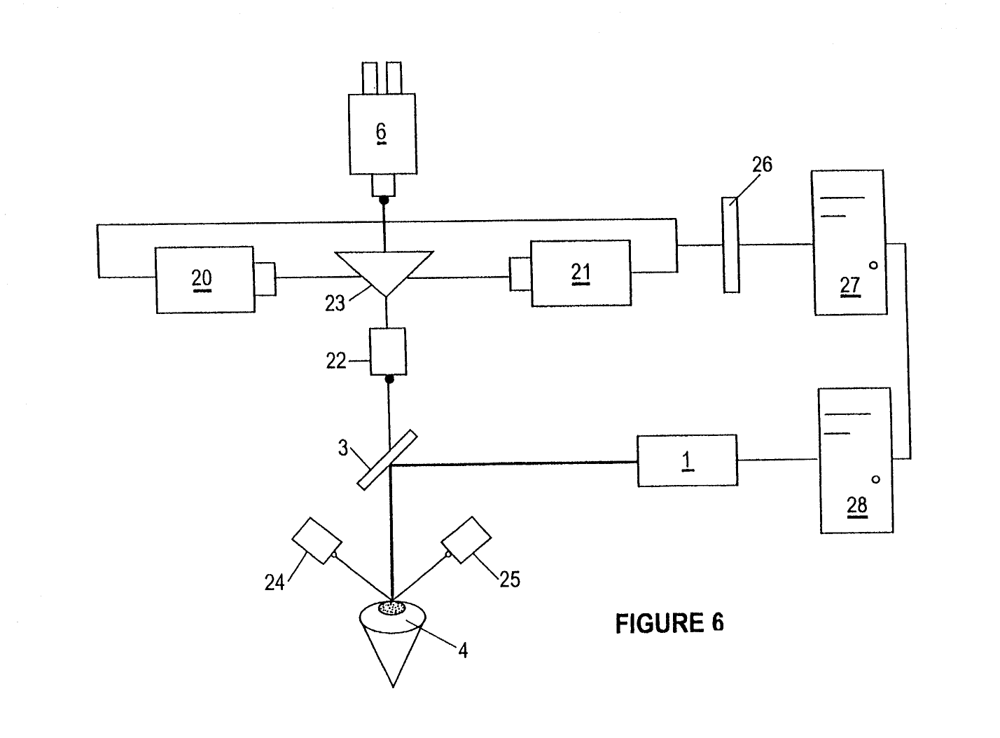

FIG. 5 depicts two images representative of steps in the analysis process, utilising the structured beam of FIG. 4; and

FIG. 6 is a view similar to FIG. 1 of an alternative embodiment of the present invention.

### PREFERRED EMBODIMENTS

Referring to FIG. 1, the medical eye-tracking device of the present invention is preferably contained within the body of a surgical laser system. A surgical laser source 1, preferably an excimer laser at 193 nm, a frequency converted solid state laser or any other laser emitting a wavelength useful for eye surgery, directs a surgical laser beam 2, incident off laser mirror 3 (which is transparent to infra-red and visible light) onto the cornea of an eye 4 to be treated. To ensure that gross eye movements are restricted, the patient's gaze is fixated on a flashing LED 5 located beneath a surgical microscope 6 with which the surgeon views the procedure. To compensate for decentration of the pupil through head, eye or saccadic movement, or a loss of patient fixation, there is provided an eye tracking system generally indicated at 30.

System 30 includes an infra-red light source 7 that generates a collimated infra-red beam 8. Beam 8 preferably passes through a diffractive beam splitter unit 32 that splits the beam into a structured beam 8′ comprising a plurality of light beam components. This beam 8′ in turn is directed, by means of first and second beamsplitters 9 and 10, towards the eye 4. Alternatively, beamsplitter 9 may be omitted and the infra-red source and unit 32 positioned in front of an imaging device. This source 7 may be a broad illumination source, preferably a LED in the form of an infra-red light, a low power laser diode, or a light with an infra-red filter.

In one preferred embodiment, one or more (in this case two) additional infra-red light sources, 11 and 12, are positioned to provide even illumination for the eye 4 from lateral directions off the optical axis defined by beams 2 and 8′ in front of the eye. Each of these sources 11 and 12, may be broad illumination sources, such as a single LED. A ring of LEDs may be substituted. Alternatively, different wavelength infra-red sources may be provided, and band pass filters used to separate the reflections. This enables images to be separated electronically for simpler and faster processing.

Three examples of suitable forms of beam 8′ are depicted in FIGS. 2 to 4, shown projected onto eye 4. These comprise a rectangular grid 16 of linear light beam components 16a, a cross 17 of two intersecting linear light beam components 17a, and a square array 18 of light spots 18a. It will be seen that, in each case, the light beam components 16a, 17a or 18a define an area of incidence on the eye substantially larger than the pupil 4a.

Light reflected from eye 4 is directed back through mirror 3, is deflected by mirror 10 and transmitted though mirror 9 to an imaging device in the form of CCD camera 3, where the reflected image is received and recorded. The image frames from the CCD camera 13 are picked up by a frame grabber 14, which digitises consecutive images, pixel by pixel, and sends information concerning each pixel to an analyser/controller in the form of a computer or microprocessor 15. This computer may also control the function of the laser 1, though this control may be provided by a separate computer.

With reference to the structured beam 18 of FIG. 4, any spot 18a that is incident on the pupil is reflected off the retina and produces a “bright-eye” reflection 35 in the reflected image 34 (FIG. 5,A). The other spots are reflected by the iris 4b and so produce “dark eye” reflections 36. The computer 15 analyses image 34 to identify which spot is incident on the pupil, and then further analyses the image on the basis of this identification to determine the location of the pupil and therefore the position or attitude of the eye. An exemplary analysis sequence includes the following steps:

for each spot, examine the variance and intensity in an annular region around the spot location (FIG. 5,B);

the annular region with the lowest variance and highest intensity is assumed to be inside the pupil;

the calculation window is reduced to a 2R×2R box centred on the spot that is inside the pupil;

the centre of the pupil is then found using a pattern matching technique, limited to the calculation window.

Where light sources 11, 12 are also included, sources 7, 11 and 12 are timed such that an image of a bright pupil reflection and a dark pupil reflection will be consecutively produced. A bright eye effect is produced as infra-red light from source 7 enters the pupil and is reflected off the retina. Light that hits the eye off axis to its centre, from sources 11 and 12, does not enter the pupil and reflect back to the camera, hence a dark pupil image is generated. Infra-red source 7 and sources 11 and 12 flash alternatively every half frame or full frame, so that consecutive half or full frames are either a “bright eye” image or a “dark eye” image. These “bright eye” and “dark eye” reflections travel sequentially via beam splitters 10 and 9 to CCD camera 13.

Image processing by custom software is carried out through controller 15 to extract the pupil from the “bright-eye” and “dark eye” image frames in order to find the pupil centre to supplement the earlier described analysis. In this supplementary technique, the images are first smoothed to reduce background noise. The “dark eye” image is then subtracted from the “bright eye” image. Thresholding is carried out to transform the resultant difference image from greyscale into black and white. A centre of mass calculation is carried out on the remaining blob and the x and y co-ordinates are calculated to find the centre of the pupil. This occurs for every frame or every second frame sent by CCD camera 13.

Finding the pupil centre by these techniques enables the tracking device to follow movement of the target region of the eye. Any deviation of the pupil centre will alert the laser controller 15. If the movement is within a certain limit, controller 15 will instruct the laser 1 to move its optics to compensate. If the movement is large, the controller will issue a warning and stop the laser until fixation is once again achieved.

In an alternative embodiment (not shown), two COD cameras are provided to directly image the eye. Two LEDs are centred in front of the lens of each camera. The imaging devices are positioned on either side of the eye to illuminate the pupil and produce the “bright eye”, “dark eye” effect. Image processing to extract the pupil is carried out as described above.

A further embodiment, illustrated in FIG. 6, involves the use of two CCD cameras 20 and 21. The two cameras are placed perpendicular to the visual axis of the eye 4, and image the eye through a prism beamsplitter 23′. The eye is illuminated by two sets of light sources. A single infra-red light source 22, preferably an infra-red LED, a low power laser diode or a light with an infra-red filter, is situated co-axial to the filter, either in front of prism beamsplitter 23 or in front of camera 20 or 21. This source 22′ may have a diffractive beamsplitter unit in front of it similar to unit 32. Two separate infra-red light sources of different wavelengths in the form of infra-red LEDs 24 and 25 are also provided on either side of the eye 4. Alternatively, a ring of infra-red LEDs may be used. Wavelength or band pass filters are used so that light from first light source 22 is only imaged by camera 20 and that light from second infra-red sources 24 and 25 is only imaged by camera 21. Thus, one camera will image a “bright eye”, the other a “dark eye”. Using electronic circuitry, the two images can be subtracted before reaching frame grabber 26. This increases the speed of the grabbing and processing. The image is then sent from frame grabber 26 to controller 27 for further processing. Connected controllers 27 and 28, for controlling image processing and laser function, are also provided.

The use of multi-component structured beams 16, 17 or 18 enlarges the possible tracking distance. If only a single spot is used instead of a structured pattern, the tracking distance is reduced to±R where R is the radius of the pupil. If the eye moves beyond this distance, the collimated light source falls onto the iris and a bright eye is not formed. The structured pattern increases the tracking distance to A+R where A is the diameter of the pattern.

In a still further embodiment, the light sources 11, 12 may be omitted and their role played instead by the general illumination lamps for the surgical procedure. These are generally off-axis and are typically broad-spectrum halogen lamps having a sufficient infra-red component to form a dark-eye image at camera 13.

Thus, the present invention provides a non-invasive eye-tracking device that is inexpensive and simple to incorporate into an existing laser system. However, it is to be understood that the invention is not limited to the particular embodiments described as examples hereinabove.

1 A method of determining the position of the pupil of an eye, including the steps of:
directing an eye-safe light beam having a plurality of components onto the eye, said components defining an area of incidence for the beam on the eye substantially larger than the pupil of the eye;
receiving an image of light thereafter reflected by the eye;
analysing the image by identifying which of the light beam components produces a bright eye reflection in said image;
determining the position of the pupil by further analysing image on the basis of said identification.

2 A method according to claim 1 wherein said eye-safe light beam defines a device comprising a pair of crossed lines that form said plurality of components.

3 A method according to claim 1 wherein said eye-safe light beam defines a grid of intersecting lines that form said plurality of components.

4 A method according to claim 1 wherein said eye-safe light beam is an array of light spots that form said plurality of components.

5 A method according to claim 1 further including generating said eye-safe light beam having a plurality of light beam components by splitting an initial light beam into said components.

6 A method according to claim 5 wherein said initial light beam and said light beam components are collimated.

7 A method according to claim 5 wherein said eye-safe light beam defines a device comprising a pair of crossed lines that form said plurality of components.

8 A method according to claim 5 wherein said eye-safe light beam is an array of light spots that form said plurality of components.

9 A method according to claim 1 wherein said further analysis is effected utilising image data for a calculation window centred on said identified component.

10 A method according to claim 9 wherein said eye-safe light beam defines a device comprising a pair of crossed lines that form said plurality of components.

11 A method according to claim 9 wherein said eye-safe light beam is an array of light spots that form said plurality of components.

12 A method according to claim 1, further including:
directing at least one further eye-safe light beam onto said eye from a lateral direction; and
receiving a further dark-eye image of light thereafter formed by reflection of said further light beam(s) by the eye;
wherein said further analysis includes subtracting or otherwise comparing said images.

13 A method according to claim 8 wherein there are a plurality of said further beams directed from different lateral directions.

14 A method according to claim 1 further including recording said image(s).

15 A method according to claim 1, including repeating said steps to monitor changes in the position of the pupil of said eye.

16 A method for controlling the aim of a surgical laser including determining the position of the pupil of an eye according to claim 1, and further including controlling said aim in response to said determined position.

17 A method according to claim 16 wherein said eye-safe light beam defines a device comprising a pair of crossed lines that form said plurality of components.

18 A method according to claim 16 wherein said eye-safe light beam is an array of light spots that form said plurality of components.

19 A method according to claim 16 further including generating said eye-safe light beam having a plurality of light beam components by splitting an initial light beam into said components.

20 A method according to claim 16, further including:
directing at least one further eye-safe light beam onto said eye from a lateral direction; and
receiving a further dark-eye image of light thereafter formed by reflection of said further light beam(s) by the eye;
wherein said further analysis includes subtracting or otherwise comparing said images.

21 A method according to claim 16, including repeating said steps to monitor changes in the position of the pupil of said eye.

22 A method according to claim 1 wherein said eye-safe beam is a beam of infra-red light.

23 Apparatus for determining the position of the pupil of an eye, including:
means for directing an eye-safe light beam having a plurality of components onto an eye, said components defining an area of incidence for the beam on the eye substantially larger than the pupil of the eye;
means for receiving an image of light thereafter reflected by the eye;
means for analysing the image by identifying which of the light beam components produces a bright eye reflection in said image, and for further analysing the image on the basis of such identification, whereby to determine the position of the pupil.

24 Apparatus according to claim 23, wherein said eye-safe light beam defines a device comprising a pair of crossed lines that form said plurality of components.

25 Apparatus according to claim 23, wherein said eye-safe light beam defines a grid of intersecting lines that form said plurality of components.

26 Apparatus according to claim 23, wherein said eye-safe light beam is an array of light spots that form said plurality of components.

27 Apparatus according to claim 23 including means for generating an eye-safe light beam and optical means for splitting the beam into said plurality of components.

28 Apparatus according to claim 27 wherein said light beam and said components are collimated.

29 Apparatus according to claim 27, wherein said eye-safe light beam defines a device comprising a pair of crossed lines that form said plurality of components.

30 Apparatus according to claim 27, wherein said eye-safe light beam is an array of light spots that form said plurality of components.

31 Apparatus according to claim 23, further including:
means for directing at least one further eye-safe light beam onto said eye from a lateral direction, said image receiving means also being arranged to receive a further dark-eye image of light formed by reflection of said further beam(s) by the eye;
wherein said further analysis includes subtracting or otherwise comparing said images;
said analysing means also comparing said images as part of said further analysis.

32 Apparatus according to claim 31 wherein there are a plurality of said further beams directed from different lateral positions.

33 Apparatus according to claim 23 wherein said image receiving means includes means to record the image(s).

34 Apparatus according to claim 23 wherein said eye-safe light is infra-red light.

35 Surgical laser apparatus including laser means for producing a beam of ablative radiation and aiming the beam at an eye, and apparatus according to claim 23 for determining the position of the pupil of said eye and for controlling the aim of said beam of ablative radiation in response to said determined position.

36 Apparatus according to claim 35, wherein said eye-safe light beam defines a device comprising a pair of crossed lines that form said plurality of components.

37 Apparatus according to claim 35, wherein said eye-safe light beam is an array of light spots that form said plurality of components.

38 Apparatus according to claim 35 including means for generating an eye-safe light beam and optical means for splitting the beam into said plurality of components.

39 Apparatus according to claim 35, further including:
means for directing at least one further eye-safe light beam onto said eye from a lateral direction, said image receiving means also being arranged to receive a further dark-eye image of light formed by reflection of said further beam(s) by the eye;
wherein said further analysis includes subtracting or otherwise comparing said images;
said analysing means also comparing said images as part of said further analysis.

40 Apparatus for facilitating determination of the position of the pupil of an eye, including:
means for generating an eye-safe light beam;
optical means disposed to receive said light beam for splitting it into a plurality of light beam components to be directed onto an eye, said components defining an area of incidence for the beam on the eye substantially larger than the pupil of the eye; and
means for receiving an image of light thereafter reflected by the eye;
wherein said light beam defines a device comprising one or more devices selected from a pair of crossed lines, a grid of intersecting lines, and an array of light spots that respectively form said plurality of components.

41 Apparatus according to claim 40 wherein said light beam and said components are collimated.

42 Apparatus according to claim 40, further including:
means for directing at least one further eye-safe light beam onto said eye from a lateral direction, said image receiving means also being arranged to receive
a further dark-eye image of light reflected from the eye after incidence of said further light beam(s).

43 Apparatus according to claim 42 wherein there are a plurality of said further beams directed from different lateral positions.

44 Apparatus according to claim 40 wherein said image receiving means includes means to record the image(s).

45 Apparatus according to claim 40 wherein said eye-safe light is infra-red light.

46 Surgical laser apparatus including laser means for producing a beam of ablative radiation and aiming the beam at an eye, and apparatus according to claim 42 for facilitating determination of the position of the pupil of said eye and for controlling the aim of said beam of ablative radiation in response to said determined position.

47 Apparatus according to claim 46, further including:
means for directing at least one further eye-safe light beam onto said eye from a lateral direction, said image receiving means also being arranged to receive a further dark-eye image of light reflected from the eye after incidence of said further light beam(s).

48 Apparatus according to claim 46 wherein said image receiving means includes means to record the image(s).

49 An apparatus for determining the position of the pupil of an eye during refractive surgery so that the aim of an ablative laser can be adjusted accordingly, including:
laser means for producing a beam of ablative radiation;
a first infra-red light source for illuminating the pupil of said eye;
an optical system for directing said first infra-red source;
a second infra-red light source for illuminating the iris of said eye;
focussing means for focussing light reflected from surfaces of the eye;
recording means for recording images of the light reflected from said surfaces;
image processing means for determining the position of the pupil of the eye; and
controller means for directing said laser to the appropriate position on the cornea in accordance with the determined position of the pupil.

50 Apparatus according to claim 49 wherein said first infra-red light source is a collimated light source.

51 A method for determining the position of the pupil of an eye during ablative laser surgery so that the aim of the ablative laser can be adjusted accordingly, including:
directing a first beam of infra-red light from a first position onto said eye;
directing at least one second beam of infra-red light from a second position onto said eye through an optical system;
imaging light from said first and second beams reflected from said eye to form first and second images respectively; and
comparing said images to determine said position of the pupil of said eye.
